(19) United States
(12) Patent Application Publication
(10) Pub. No.: US 2018/0197425 A1
(43) Pub. Date: Jul. 12, 2018

(54) SELF-MONITORING ANALYSIS AND REPORTING TECHNOLOGIES

(71) Applicant: Washington State University, Pullman, WA (US)

(72) Inventor: Richard Lamb, Buffalo, NY (US)

(21) Appl. No.: 15/400,760

(22) Filed: Jan. 6, 2017

(51) Int. Cl. G09B 5/12; G09B 5/08; G09B 5/14; A61B 5/00; A61B 5/11; A61B 5/16

(52) U.S. Cl. CPC G09B 5/12; G09B 5/08; G09B 5/14; A61B 5/7267; A61B 5/1123; A61B 5/165; A61B 5/6897; A61B 5/0077

(57) ABSTRACT

Self-monitoring analysis and reporting techniques are described. Within an educational environment, a teacher computer and one or more student workstations are communicatively connected via a network. The student workstations are implemented to include one or more sensors and a neural network to analyze data from the sensors to determine a cognitive or affective state of the student. Based on the state of the student, a student user interface can be updated to maintain the student's attention and/or to prompt the student to seek out help or additional resources when confused. Furthermore, based on the state of the student, a teacher user interface can be updated to provide real-time visualization of the current state of each student in the educational environment.

### BACKGROUND

Although a considerable amount of testing occurs within schooling environments (e.g., K-12 or college), there is little use of test data for educational decision-making and modification of classroom instruction. Typical classroom environments include a single teacher and many students (e.g., 15-30 or more in a K-12 setting, possibly many more in a college environment). Teachers are expected to teach material while constantly evaluating the degree to which individual students are engaged and comprehending the material being presented.

### SUMMARY

This disclosure describes systems and methods for self-monitoring analysis and reporting in an educational setting. In at least one example, an educational environment (e.g., a classroom) includes a teacher computer with display and one or more student workstations equipped with a student computer and one or more sensors. Sensors gather any combination of keystroke data, mouse click data, movement data, facial expression data, physiological data, neuroimaging data, or other biometric and autonomic nervous system data. Based on the received sensor data, the student computer uses a neural network (machine learning algorithm) to determine and classify cognitive or affective states of a student. A student user interface may be modified based on the determined cognitive or affective states of the user. For example, the computer may prompt the user to pay attention when the user is determined to be inattentive, or to seek additional help when the student is determined to be confused. In addition, data indicating the student state is transmitted to the teacher computer to enable display of a teacher user interface that includes representations of each student workstation within the classroom, and an indication of a current cognitive or attentive state of each student using each respective student workstation.

This Summary is provided to introduce a selection of concepts in a simplified form that are further described below in the Detailed Description. This Summary is not intended to identify key or essential features of the claimed subject matter, nor is it intended to be used as an aid in determining the scope of the claimed subject matter. The term “techniques,” for instance, may refer to system(s), method(s), computer-readable instructions, module(s), algorithms, hardware logic, and/or operation(s) as permitted by the context described above and throughout the document.

### BRIEF DESCRIPTION OF THE DRAWINGS

The detailed description is described with reference to the accompanying figures. In the figures, the left-most digit(s) of a reference number identifies the figure in which the reference number first appears. The same reference numbers in different figures indicate similar or identical items.

FIG. 1 is a block diagram illustrating an example data flow using example self-monitoring analysis and reporting technologies.

FIG. 2 is a block diagram illustrating an example environment in which the example self-monitoring analysis and reporting technologies can be implemented.

FIG. 3 is a pictorial diagram illustrating an example desk with example sensors implemented as a component of a student workstation.

FIG. 4 is a pictorial diagram illustrating an example chair with example sensors implemented as a component of a student workstation.

FIG. 5 is a pictorial diagram illustrating an example series of user interface components that may be presented at a student workstation.

FIG. 6 is a pictorial diagram illustrating example user interface components that may be presented at a teacher computer within an educational environment.

FIG. 7 is a block diagram illustrating select components of an example student computer.

FIG. 8 is a block diagram illustration select component of an example teacher computer.

FIG. 9 is a flow diagram of an example method for implementing self-monitoring analysis and reporting technologies at a student computer.

FIG. 10 is a flow diagram of an example method for implementing self-monitoring analysis and reporting technologies at a teacher computer.

### DETAILED DESCRIPTION

### Overview

Examples of self-monitoring analysis and reporting technologies described herein provide real-time feedback to both students and teachers to enable accurate and timely understanding of student learning and effectiveness of instruction. Biometric sensor data in conjunction with facial expression, mouse click, keystroke, and other forms of traditional educational measurement data provides an indication of student affective and cognitive states.

The gathered data can be used to determine when to prompt a student to pay attention or to request additional information or help in addition to providing additional content for educational purposes. Furthermore, the gathered data can also be used to indicate to the teacher when individual students are, for example, confused or disengaged.

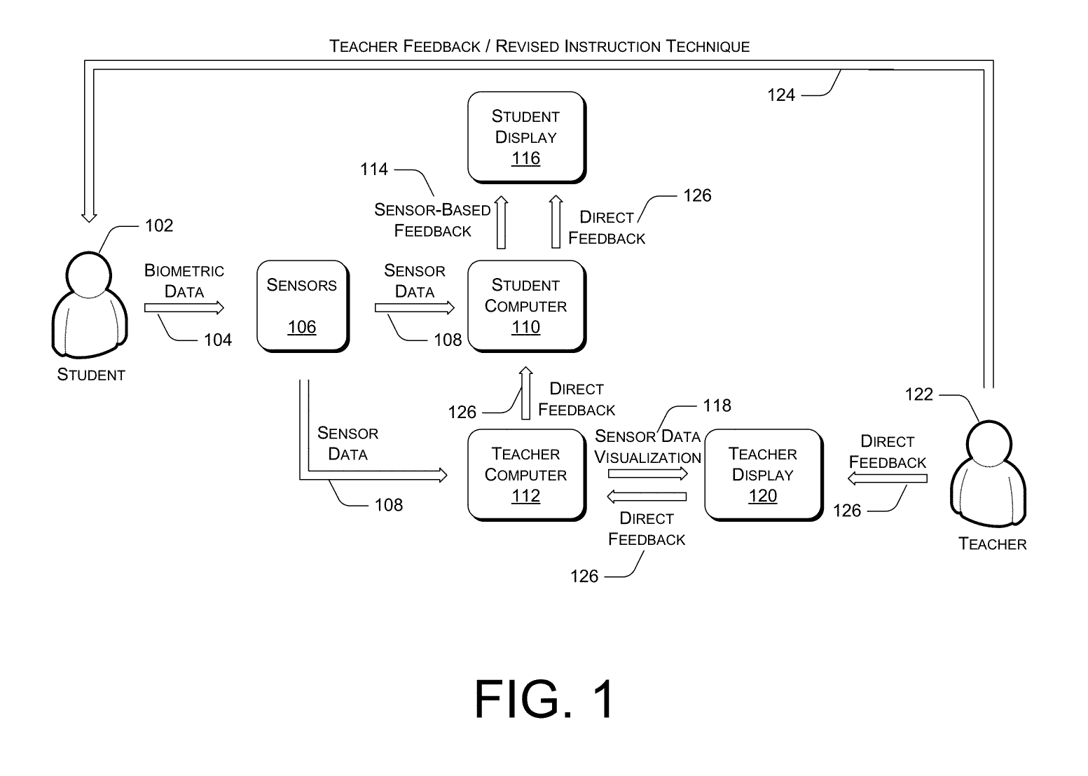

FIG. 1 illustrates an example data flow using example self-monitoring analysis and reporting technologies as described herein. In the illustrated example, a student 102 passively provides biometric data 104 to one or more sensors 106. For example, the chair in which the student is sitting and/or the desk at which the student is sitting may include any number of biometric sensors to record, for example, heart rate, respiration, blood pressure, and galvanic skin response data. Based on the received biometric data 104, sensors 106 provide sensor data 108 to both the student computer 110 and the teacher computer 112. Sensor data 108 may, for example, indicate that the student is appropriately engaged in the current lesson, that the student is in a state of confusion, or that the student is disengaged or otherwise not paying attention.

Depending on the received sensor data 108, student computer 110 may provide sensor-based feedback 114 via student display 116. For example, if the sensor data 108 indicates that the student is not paying attention, the sensor-based feedback 114 may include a prompt directing the student to pay attention. As another example, if the sensor data 108 indicates that the student is confused, the sensor-based feedback 114 may include a prompt to ask the student if they would like to ask a question or access additional practice examples or other resources.

In response to the received sensor data 108, teacher computer 112 provides a sensor data visualization 118 via teacher display 120. In an example, the sensor data visualization 118 may provide a visual indicator of a degree of attentiveness, confusion, or inattentiveness associated with the student based on the sensor data.

Upon viewing the sensor data visualization, the teacher 122 may provide feedback and/or revise their instruction technique, as indicated by arrow 124. Alternatively, the teacher may provide direct feedback 126 (e.g., a textual message to the student) via the teacher display 120. The direct feedback 126 may be transmitted from the teacher computer 112 to the student computer 110 for viewing by the student 102 via the student display 116. In addition, the student computer 110 may provide autonomous responses to the student to assist in reducing student confusion.

### Illustrative Environment

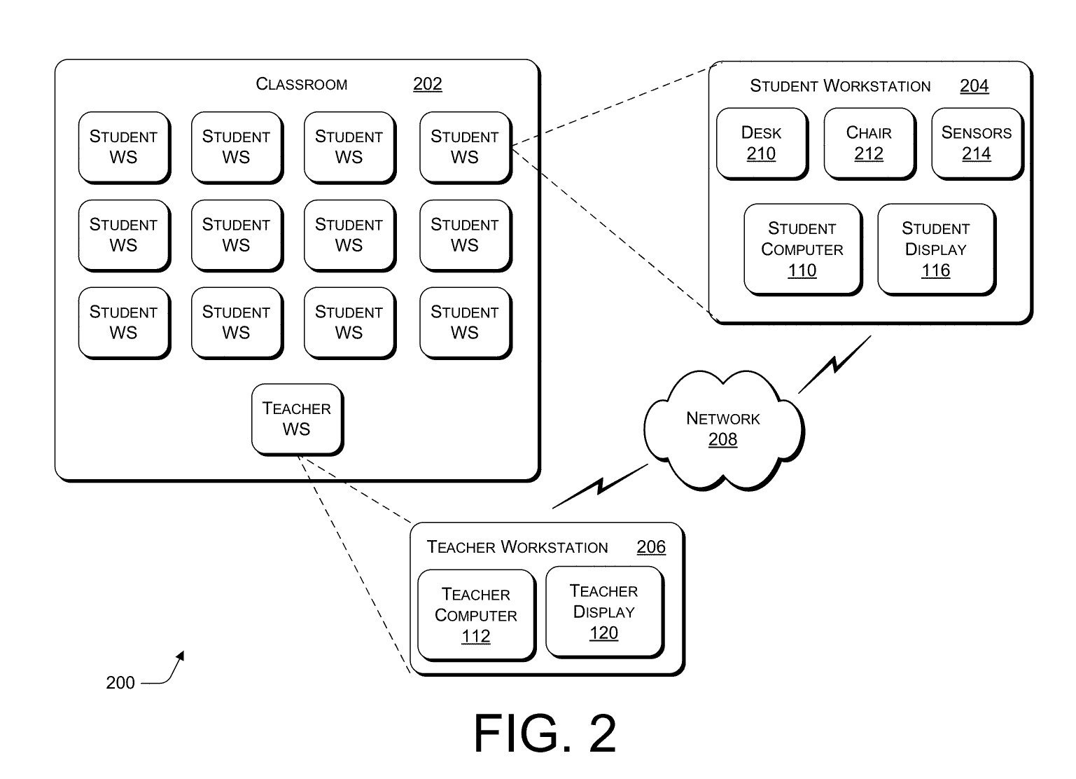

FIG. 2 shows an example environment 200 in which examples of self-monitoring analysis and reporting technologies can operate. Example environment 200 is illustrated as an educational classroom 202, which includes multiple student workstations 204 and a teacher workstation 206. In some examples, the various devices and/or components of environment 200 communicate with one another, and may communicate with external devices via one or more networks 208.

Each student workstation 204 includes a desk 210, a chair 212, one or more sensors 214, a student computer 110, and a student display 116. In an example implementation, sensors 214 are attached to, or implemented as components of, the desk 210 and/or the chair 212. Student computer 110 and student display 116 may be implemented as components of a single device, such as a laptop computer, or as separate components communicatively connected to one another.

Teacher workstation 206 includes a teacher computer 112 and a teacher display 120. Teacher computer 112 and teacher display 120 may be implemented as components of a single device, such as a laptop computer, or as separate components communicatively connected to one another.

Network 208 can include, for example, public networks such as the Internet, private networks such as an institutional and/or personal intranet, or some combination of private and public networks. Network 208 can also include any type of wired and/or wireless network, including but not limited to local area networks (LANs), wide area networks (WANs), satellite networks, cable networks, Wi-Fi networks, WiMax networks, mobile communications networks (e.g., 3G, 4G, and so forth) or any combination thereof. Network 208 can utilize communications protocols, including packet-based and/or datagram-based protocols such as internet protocol (IP), transmission control protocol (TCP), user datagram protocol (UDP), or other types of protocols. Moreover, network 208 can also include a number of devices that facilitate network communications and/or form a hardware basis for the networks, such as switches, routers, gateways, access points, firewalls, base stations, repeaters, backbone devices, and the like.

In some examples, network 208 can further include devices that enable connection to a wireless network, such as a wireless access point (WAP). Examples support connectivity through WAPs that send and receive data over various electromagnetic frequencies (e.g., radio frequencies), including WAPs that support Institute of Electrical and Electronics Engineers (IEEE) 802.11 standards (e.g., 802.11g, 802.11n, and so forth), and other standards.

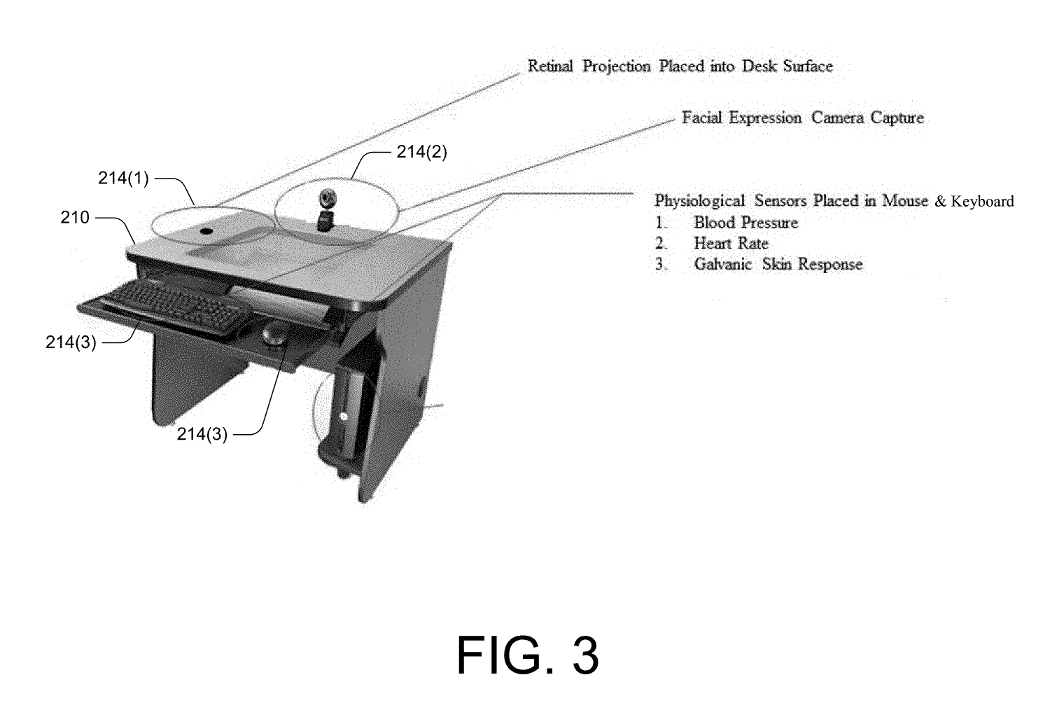

FIG. 3 illustrates an example desk 210 of a student workstation 204, fitted with a plurality of sensors 214. For example, a desk 210 may be fitted with any combination of a retinal projection 214(1), a camera 214(2) to capture facial expressions, and physiological sensors 214(3) in or near a mouse and/or a keyboard, such as a blood pressure sensor, a heart rate sensor, and a galvanic skin response sensor.

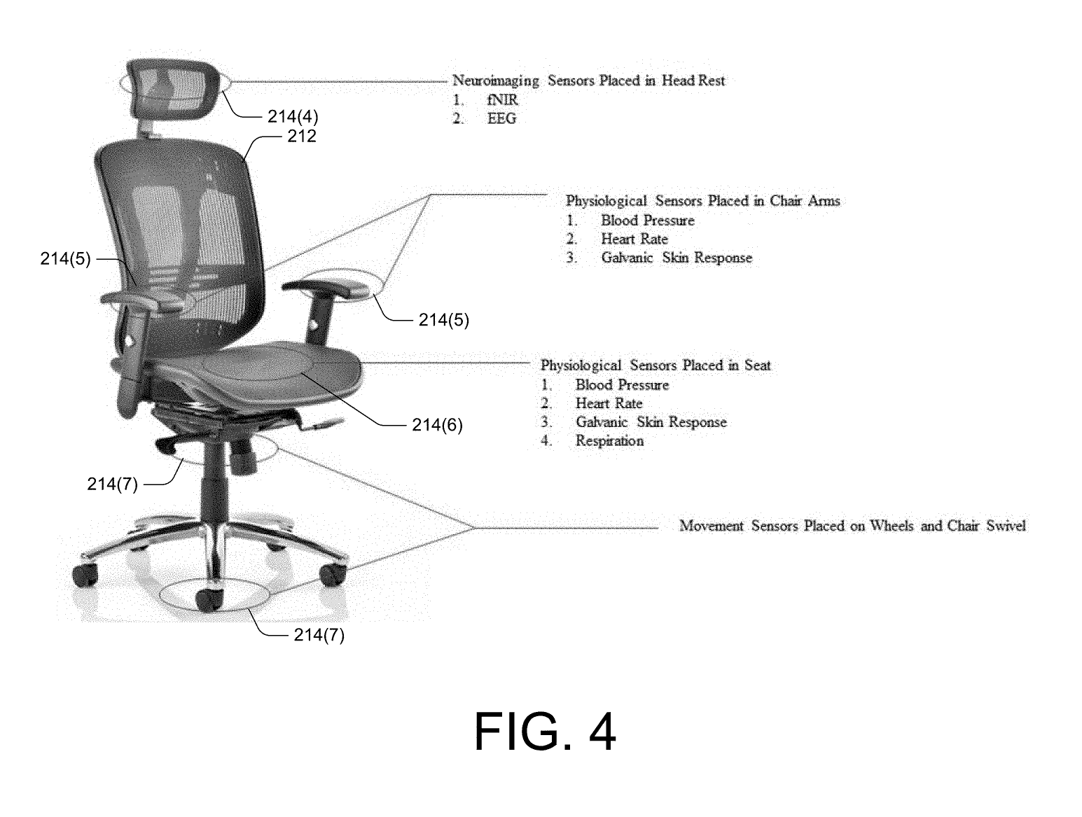

FIG. 4 illustrates an example chair 212 of a student workstation 204, fitted with a plurality of sensors 214. For example, a chair 212 may be fitted with any combination of neuroimaging sensors 214(4), physiological sensors 214(5) and 214(6), and movement sensors 214(7). In an example implementation, neuroimaging sensors 214(4) may include a functional Near Infrared Spectroscopy (fNIRS) sensor and/or an electroencephalogram (EEG) sensor within a headrest component of the chair 212, physiological sensors 214(5) may include a blood pressure sensor, a heart rate sensor, and a galvanic skin response sensor in or on arms of the chair 212, physiological sensors 214(6) may include a blood pressure sensor, a heart rate sensor, a galvanic skin response sensor, and a respiration sensor in or on a seat of the chair 212, and movement sensors 214(7) may be placed to capture movement through wheels and/or a swivel component of the chair 212.

fNIRS is a non-invasive, save, and portable optical neuroimaging method that offers a high temporal resolution to assess cognitive dynamics and affective state during various learning tasks. fNIRS uses specific wavelengths of light to provide measures of cerebral oxygenated and deoxygenated hemoglobin. In an example implementation, an increase of oxygenated blood is interpreted as an increase in cognitive effort.

### Student Experience

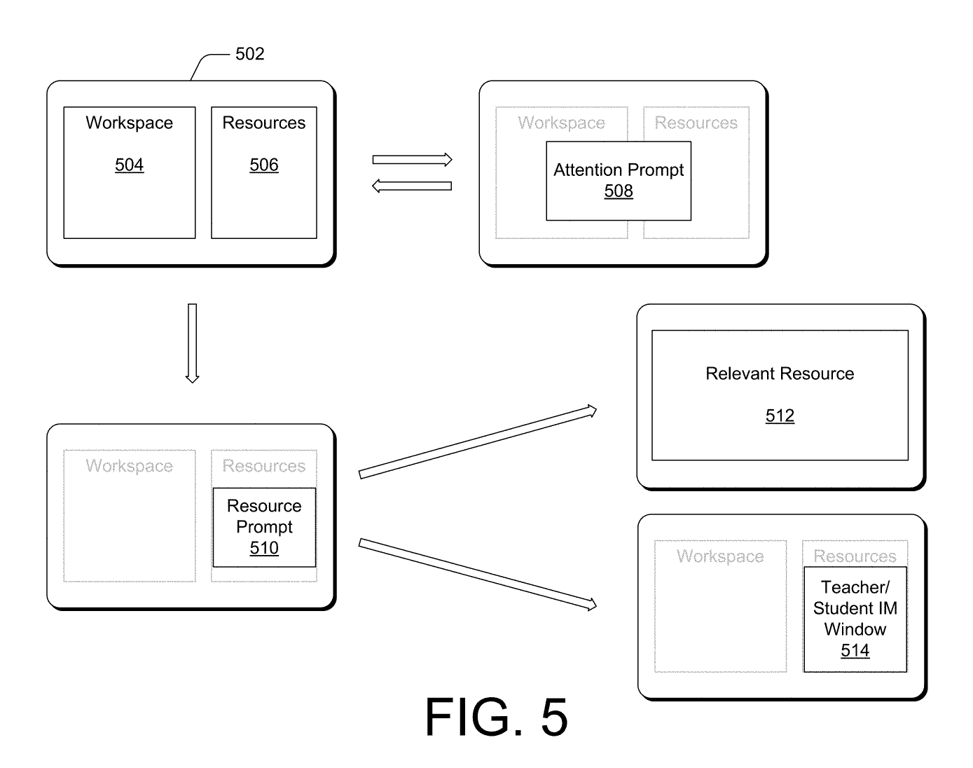

FIG. 5 illustrates an example series of user interface components that may be presented via the student display 116. In an example scenario, during a lecture by the teacher 122, student computer 110 may cause a user interface 502 to be presented via student display 116. In the illustrated example, user interface 502 includes a workspace area 504 and a resources area 506. Workspace area 504 may present practice problems or homework problems for a student to work on, while resources area 506 may present examples, notes, links to online resources, and so on. Workspace area 504 and/or resources area 506 may be active or inactive at various times based, for example, on data received from teacher computer 112. For example, a teacher may choose to deactivate the workspace area during a portion of a lecture, and activate the workspace area when the students are to solve a practice problem during an interactive portion of the lecture.

As described above with reference to FIGS. 1-4, sensors at the student workstation 204 capture data such as biometric data, facial expressions, mouse clicks, key strokes, chair movements, and so on, during the lecture. If the sensor data indicates that the student is disengaged or otherwise not paying attention, user interface 502 may display an attention prompt 508. In an example implementation, attention prompt 508 may be brightly colored, may flash or blink, or may otherwise be configured to capture the student's attention. In an example, a mouse click on the attention prompt may dismiss the prompt, indicating that the student's attention has been re-established.

In addition to detecting that a student is disengaged, sensor data may also be used to determine that a student is confused. In an example scenario, there may be times when the student is expected to perform various practice or homework problems within the workspace area 504. While the student is working within the workspace area 504, if the sensor data indicates a state of student confusion, a resource prompt 510 may be displayed. In the illustrated example, resource prompt 510 may enable the student to access help through a relevant resource 512 or through direct communication with the teacher, for example, through an instant messaging widow 514. In various implementations, a resource prompt 510 may allow a student to choose between a link to a relevant resource 512 or an IM window 514. Alternatively, resource prompt 510 may be a prompt to access relevant resource 512 or a prompt to access the IM window 514, and either may be triggered depending on a degree of student confusion represented by the sensor data. For example, if the student is moderately confused, the resource prompt may include a link to a relevant resource 512, encouraging the student to explore the relevant resource 512 on their own. However, if the sensor data indicates that the student is significantly confused or disengaged, the resource prompt 510 may only provide for access to the IM window 514, encouraging the student to ask the teacher for additional assistance.

In another example, if student confusion is detected while the workspace is inactive (e.g., while the teacher is lecturing), data indicating the state of the student may be transmitted to the teacher computer, but no prompt may be presented to the student.

### Teacher Experience

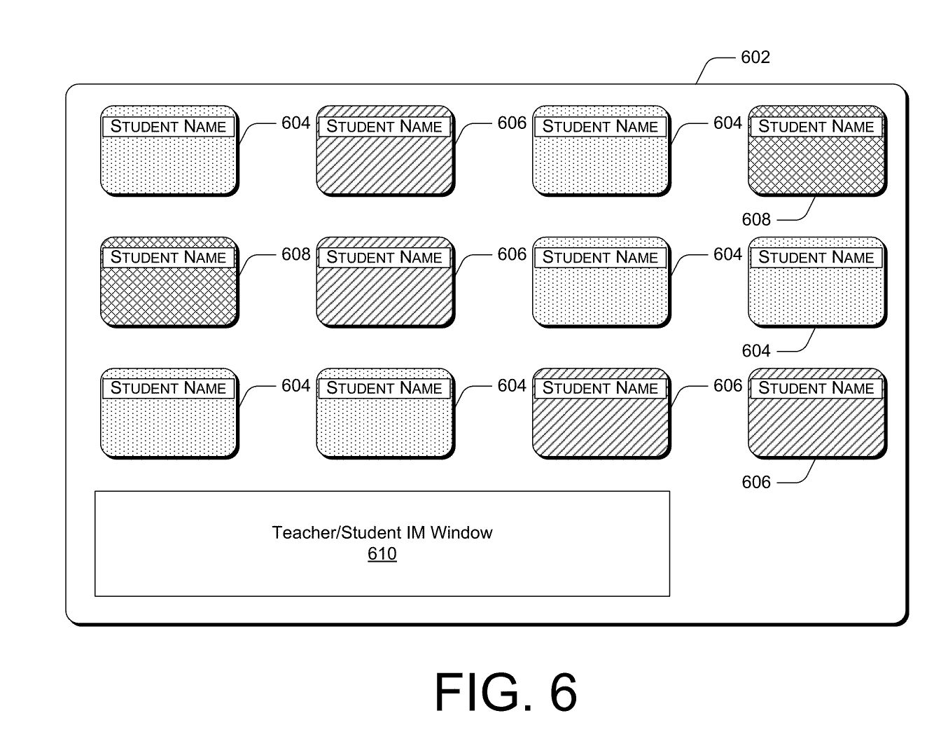

FIG. 6 illustrates an example user interface 602 that may be presented via the teacher display 120. In the illustrated example, each student workstation is indicated as a block on the user interface, for example, with the student name as a label. Each block visually indicates a detected state of the student based on the sensor data. Text, colors, shading, blinking, flashing, or any other type of visual indicator may be used to indicate a student state. For example, a green block may indicate that a student is engaged and not showing signs of confusion, a yellow block may indicate that the student is confused, and a red block may indicate that the student is disengaged. In the illustrated example, blocks 604 have a light shading, which may indicate that the student represented by the block is paying attention and not showing signs of confusion. Blocks 606 have a moderate shading, which may indicate that the student represented by the block is paying attention, but showing signs of confusion. Blocks 608 have a significant shading, which may indicate that the student represented by the block is not paying attention.

As an example, if the user interface 602 is displayed while the teacher is presenting a lesson, the teacher may, in real time and based on the user interface display, modify their presentation technique or engage directly with one or more of the students showing signs of inattentiveness or confusion. As another example, if the user interface 602 is displayed while the students are working on an assigned practice or homework problem, the teacher may, in response to the user interface display, reach out to provide one-on-one assistance to one or more of the students for whom the sensors indicate are confused or not paying attention.

In an example implementation, user interface 602 may also enable the teacher to send and/or receive messages such as through an instant messaging (IM) window 610. For example, especially useful in a large class setting, the teacher may click on the block 604, 606, or 608 representing a particular student to launch an IM window 610 for directly communicating via IM with the particular student represented by the selected block. As described above with reference to FIG. 5, students may also initiate IM sessions with the teacher, which may be displayed in IM window 610 or as an IM window overlaying the block associated with student who initiated the IM session.

### Example Computing Devices

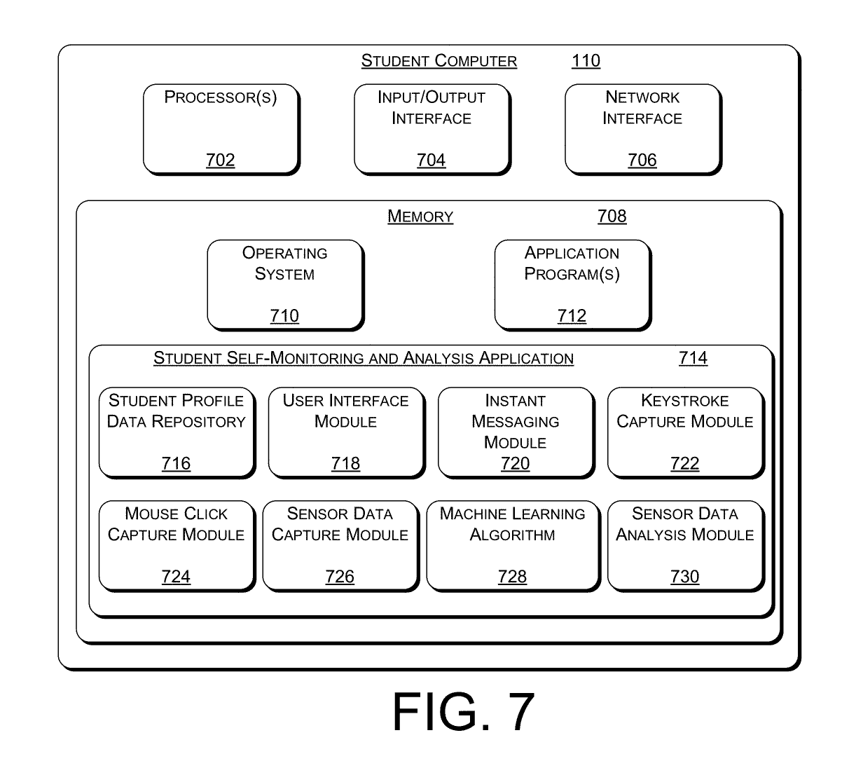

FIG. 7 illustrates select components of an example student computer 110. Example student computer 110 includes one or more processor(s) 702, input/output interface 704, network interface 706, and memory 708.

Processor(s) 702 can be implemented as, for example, a CPU-type processing unit, a GPU-type processing unit, a field-programmable gate array (FPGA), another class of digital signal processor (DSP), or other hardware logic components that may, in some instances, be driven by a CPU. For example, and without limitation, illustrative types of hardware logic components that can be used include Application-Specific Integrated Circuits (ASICs), Application-Specific Standard Products (ASSPs), System-on-a-chip systems (SOCs), Complex Programmable Logic Devices (CPLDs), etc.

Input/output interface 704 allows student computer 110 to communicate with input/output devices such as user input devices including peripheral input devices (e.g., a keyboard, a mouse, a pen, a game controller, a voice input device, a touch input device, a gestural input device, and the like) and/or output devices including peripheral output devices (e.g., a display, a printer, audio speakers, a haptic output, and the like).

Network interface 706 enables communications between student computer 110 and other networked devices such as teacher computer 112. Network interface 706 can include one or more network interface controllers (NICs) or other types of transceiver devices to send and receive communications over a network.

Processor 702 is operably connected to memory 708 such as via a bus (not shown), which in some instances can include one or more of a system bus, a data bus, an address bus, a PCI bus, a Mini-PCI bus, and any variety of local, peripheral, and/or independent buses.

Memory 708 includes operating system 710, any number of application programs 712, and student self-monitoring and analysis application 714. Operating system 710, application programs 712, and student self-monitoring and analysis application 714 are implemented as executable instructions stored in the memory 708 that are loadable and executable by processor(s) 702.

Example student self-monitoring and analysis application 714 includes student profile data repository 716, user interface module 718, instant messaging module 720, keystroke capture module 722, mouse click capture module 724, sensor data capture module 726, machine learning algorithm 728, and sensor data analysis module.

Student profile data repository 716 stores profile data associated with one or more students. For example, in a classroom setting, different students may sit at different workstations on different days. Similarly, in a high school or college setting, different classes may be held in a single classroom throughout a day, and thus, multiple students may utilize a single student workstation in a single day. Student profile data repository 716 may include student identifying information such as a student name, username, password, and so on.

User interface module 718 is configured to render student user interface components such as those illustrated in and described above with reference to FIG. 5.

Instant messaging module 720 is configured to enable instant messaging between student computer 110 and teacher computer 112. For example, as illustrated and described above with reference to FIG. 5, teacher/student IM window 514 may be initiated from within the student user interface 502.

Keystroke capture module 722 is configured to capture keystrokes entered through a keyboard associated with student computer 110. Keystroke capture module 722 may send the captured keystroke data to sensor data machine learning algorithm 728.

Mouse click capture module 724 is configured to capture data representing mouse clicks entered through a mouse associated with student computer 110. Mouse click capture module 722 may send the captured mouse click data to machine learning algorithm 728.

Sensor data capture module 726 receives sensor data from one or more sensors associated with student workstation 204. Sensor data capture module may receive neuroimaging data from an fNIR sensor and/or an EEG sensor. Sensor data capture module may receive blood pressure readings, heart rate readings, galvanic skin response readings, and/or respiration readings from physiological sensors associated with student workstation 204. Sensor data capture module may receive facial expression data captured by a camera associated with student workstation 204. Sensor data capture module may also receive motion readings from motion sensors associated with student workstation 204.

Machine learning algorithm 728 is configured to analyze received sensor data and to determine a cognitive or affective state of a student based on the received sensor data. In an example implementation, machine learning algorithm 728 is implemented as a neural network, initially trained in a supervised learning environment. For example, using respiration, heart rate, blood pressure, skin conductance, keystroke, mouse click, and facial expression data is collected in real-time and analyzed to develop a subject-independent estimation algorithm for determining student attentive and/or cognitive states.

Sensor data analysis module 730 is configured to cause user interface module 718 to modify, based on the determined cognitive or affective state of the student, a user interface being presented.

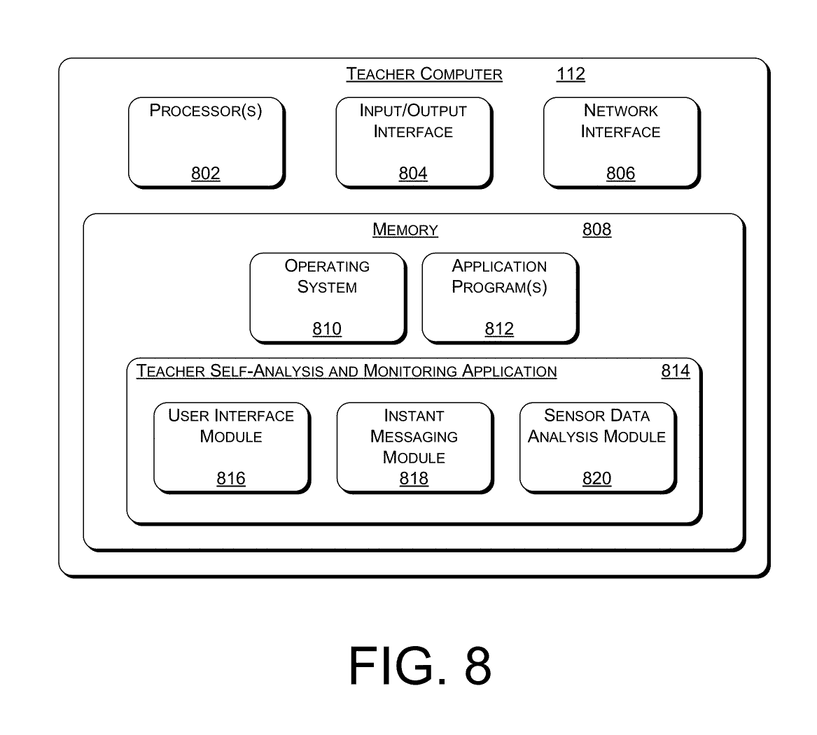

FIG. 8 illustrates select components of an example teacher computer 112. Example teacher computer 112 includes one or more processor(s) 802, input/output interface 804, network interface 806, and memory 808.

Processor(s) 802 can be implemented as, for example, a CPU-type processing unit, a GPU-type processing unit, a field-programmable gate array (FPGA), another class of digital signal processor (DSP), or other hardware logic components that may, in some instances, be driven by a CPU. For example, and without limitation, illustrative types of hardware logic components that can be used include Application-Specific Integrated Circuits (ASICs), Application-Specific Standard Products (ASSPs), System-on-a-chip systems (SOCs), Complex Programmable Logic Devices (CPLDs), etc.

Input/output interface 804 allows teacher computer 112 to communicate with input/output devices such as user input devices including peripheral input devices (e.g., a keyboard, a mouse, a pen, a game controller, a voice input device, a touch input device, a gestural input device, and the like) and/or output devices including peripheral output devices (e.g., a display, a printer, audio speakers, a haptic output, and the like).

Network interface 806 enables communications between teacher computer 112 and other networked devices such as student computer 110. Network interface 806 can include one or more network interface controllers (NICs) or other types of transceiver devices to send and receive communications over a network.

Processor 802 is operably connected to memory 808 such as via a bus (not shown), which in some instances can include one or more of a system bus, a data bus, an address bus, a PCI bus, a Mini-PCI bus, and any variety of local, peripheral, and/or independent buses.

Memory 808 includes operating system 810, any number of application programs 812, and teacher self-monitoring and analysis application 814. Operating system 810, application programs 812, and teacher self-monitoring and analysis application 814 are implemented as executable instructions stored in the memory 808 that are loadable and executable by processor(s) 802.

Example teacher self-monitoring and analysis application 814 includes user interface module 816, instant messaging module 818, and sensor data analysis module 820.

User interface module 816 is configured to render teacher user interface components such as those illustrated in and described above with reference to FIG. 6.

Instant messaging module 818 is configured to enable instant messaging between teacher computer 112 and student computer 110. For example, as illustrated and described above with reference to FIG. 6, teacher/student IM window 610 may be initiated from within the teacher user interface 602.

Sensor data analysis module 820 is configured to cause user interface module 816 to modify, based on the determined cognitive or affective state of the student, a teacher user interface being presented.

Memory 708 and memory 808 are examples of computer-readable media and can store instructions executable by the processors 702 and 708. Memory 708 and/or memory 808 can also store instructions executable by external processing units such as by an external CPU, an external GPU, and/or executable by an external accelerator, such as an FPGA type accelerator, a DSP type accelerator, or any other internal or external accelerator. In various examples at least one CPU, GPU, and/or accelerator is incorporated in student computer 110 or teacher computer 112, while in some examples one or more of a CPU, GPU, and/or accelerator is external to student computer 110 or teacher computer 112.

Computer-readable media may include computer storage media and/or communication media. Computer storage media can include volatile memory, nonvolatile memory, and/or other persistent and/or auxiliary computer storage media, removable and non-removable computer storage media implemented in any method or technology for storage of information such as computer-readable instructions, data structures, program modules, or other data. Memory 708 and memory 808 can be examples of computer storage media. Thus, the memory 708 and memory 808 may include tangible and/or physical forms of media included in a device and/or hardware component that is part of a device or external to a device, including but not limited to random-access memory (RAM), static random-access memory (SRAM), dynamic random-access memory (DRAM), phase change memory (PRAM), read-only memory (ROM), erasable programmable read-only memory (EPROM), electrically erasable programmable read-only memory (EEPROM), flash memory, compact disc read-only memory (CD-ROM), digital versatile disks (DVDs), optical cards or other optical storage media, magnetic cassettes, magnetic tape, magnetic disk storage, magnetic cards or other magnetic storage devices or media, solid-state memory devices, storage arrays, network attached storage, storage area networks, hosted computer storage or any other storage memory, storage device, and/or storage medium that can be used to store and maintain information for access by a computing device.

In contrast to computer storage media, communication media may embody computer-readable instructions, data structures, program modules, or other data in a modulated data signal, such as a carrier wave, or other transmission mechanism. As defined herein, computer storage media does not include communication media. That is, computer storage media does not include communications media consisting solely of a modulated data signal, a carrier wave, or a propagated signal, per se.

### Example Processes

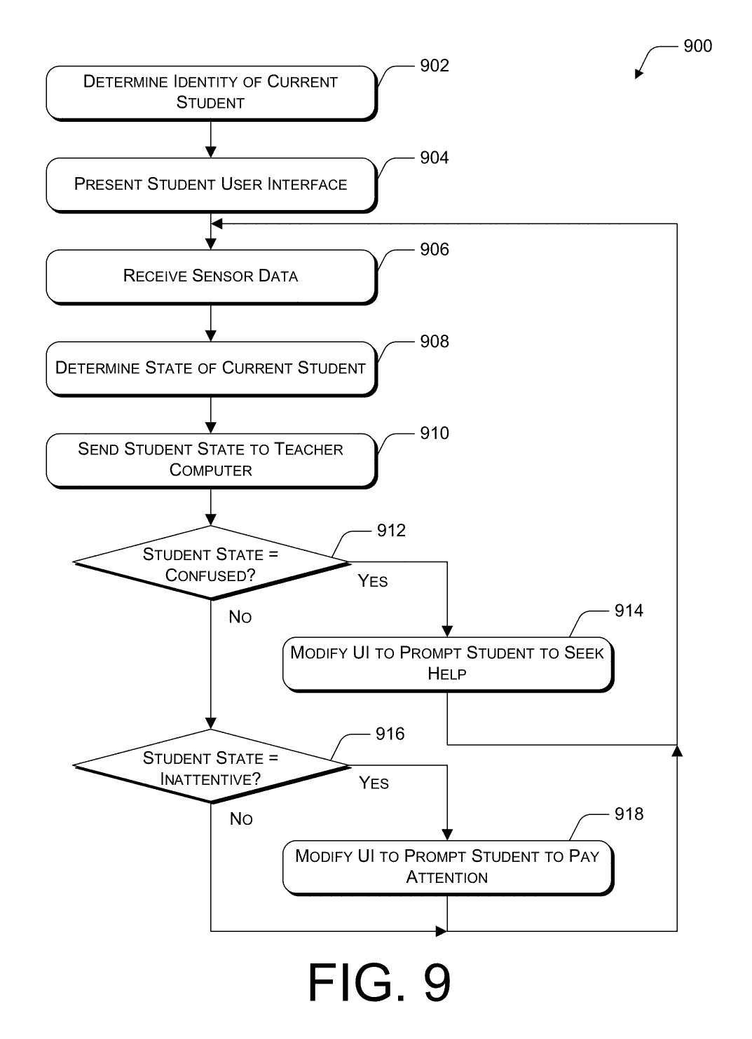

FIG. 9 illustrates an example process 900 to operate self-monitoring analysis and reporting technologies as described herein. The process is illustrated as a set of operations shown as discrete blocks. The process may be implemented in any suitable hardware, software, firmware, or combination thereof. The order in which the operations are described is not to be construed as a limitation.

At block 902, an identity of a current student is determined. For example, student self-monitoring and analysis application 718 determines a student profile stored in student profile data repository and associated with a current user of student computer 110. For example, a student profile may be determined based on login data or biometric data.

At block 904, a student user interface is presented. For example, user interface module 718 presents a student user interface 502 via student display 116.

At block 906, sensor data is received. For example, student computer 110 receives sensor data from one or more sensors via input interface 704. Sensor data may include, for example, any combination of keystroke data, mouse click data, movement data, physiological data, facial expression data, or neuroimaging data from sensors associated with the student workstation 204.

At block 908, a cognitive or affective state of the current student. For example, machine learning algorithm 728 processes the received sensor data to determine a cognitive or affective state of the current student. For example, machine learning algorithm 728 uses a neural network to analyze the received sensor data and determine the cognitive or affective state of the current student.

At block 910, the cognitive or affective state of the current user is sent to the teacher computer. For example, the cognitive or affective state of the current user, as output from the machine learning algorithm 728 is transmitted over the network 208 from student computer 110 to teacher computer 112.

At block 912, it is determined whether or not the state of the current student indicates confusion. For example, sensor data analysis module 730 analyzes the output from the machine learning algorithm 728 to determine whether or not the state of the current student indicates confusion.

If the state of the current student indicates confusion (the “Yes” branch from block 912), then at block 914, the user interface is modified to prompt the current student to seek help. For example, sensor data analysis module 730 directs user interface module 718 to modify the student user interface 502 based on the student state indicating that the student is confused. For example, sensor data analysis module 730 may direct user interface module 718 to present a prompt to suggest that the student request additional help. Processing then continues as described above with reference to block 906.

On the other hand, if the state of the current student does not indicate confusion (the “No” branch from block 912), then at block 916, it is determined whether or not the state of the current user indicates inattentiveness. For example, sensor data analysis module 730 analyzes the output from the machine learning algorithm 728 to determine whether or not the state of the current student indicates that the current student is not paying attention.

If the state of the current user indicates inattentiveness (the “Yes” branch from block 916), then at block 918 the user interface is modified to prompt the current user to pay attention. For example, sensor data analysis module 730 directs user interface module 718 to modify the student user interface 502 based on the student state indicating that the student is not paying attention. For example, sensor data analysis module 730 may direct user interface module 718 to present a prompt to refocus the student's attention. Processing then continues as described above with reference to block 906.

On the other hand, if the state of the current user does not indicate inattentiveness (the “No” branch from block 916), then processing continues as described above with reference to block 906.

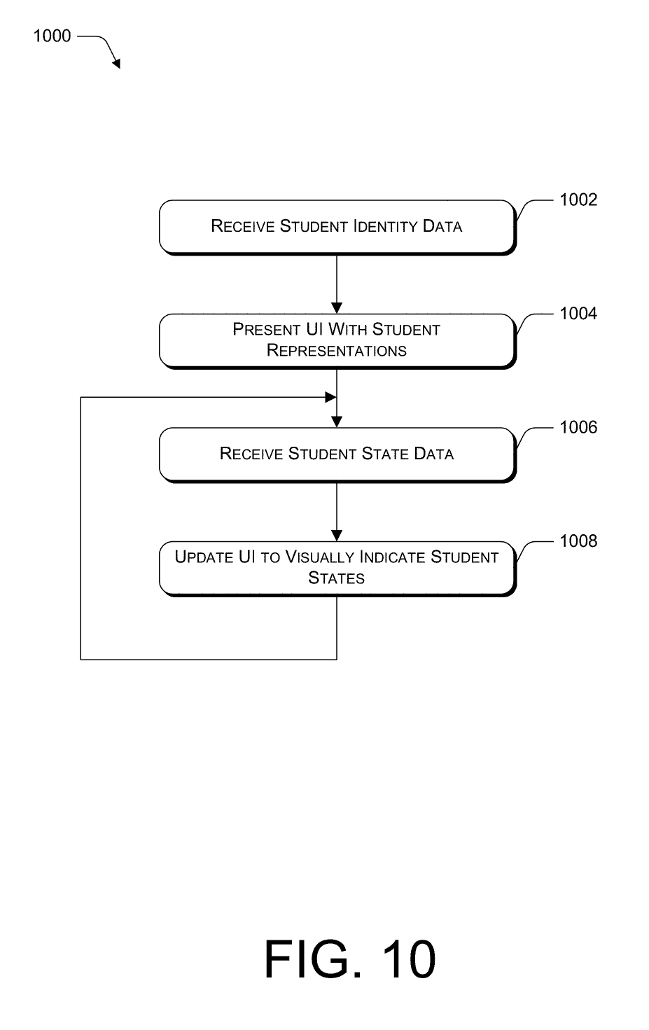

FIG. 10 illustrates an example process 1000 to operate self-monitoring analysis and reporting technologies as described herein. The process is illustrated as a set of operations shown as discrete blocks. The process may be implemented in any suitable hardware, software, firmware, or combination thereof. The order in which the operations are described is not to be construed as a limitation.

At block 1002, a student identity is received. For example, teacher computer 112 receives from student computer 110, data indicating the identity of a student currently using student computer 110. If multiple student computers are in use, the identity of each respective student using a student computer may be received.

At block 1004, a user interface with student representations is presented. For example, user interface module 816 presents a teacher user interface 602 including a representation of each student workstation 204.

At block 1006, student state data is received. For example, teacher computer 112 receives from student computer 110 data indicating a cognitive or attentive state of a student using a student computer 110.

At block 1008, the user interface is updated to indicate student attentive or cognitive states. For example, sensor data analysis module 820 analyzes the received data indicating student cognitive or attentive states, and directs user interface module 816 to modify the teacher user interface being presented to indicate, for each student workstation representation, a current state of the respective users of the student workstations.

### CONCLUSION

Although the techniques have been described in language specific to structural features and/or methodological acts, it is to be understood that the appended claims are not necessarily limited to the features or acts described. Rather, the features and acts are described as example implementations of such techniques.

The operations of the example processes are illustrated in individual blocks and summarized with reference to those blocks. The processes are illustrated as logical flows of blocks, each block of which can represent one or more operations that can be implemented in hardware, software, or a combination thereof. In the context of software, the operations represent computer-executable instructions stored on one or more computer-readable media that, when executed by one or more processors, enable the one or more processors to perform the recited operations. Generally, computer-executable instructions include routines, programs, objects, modules, components, data structures, and the like that perform particular functions or implement particular abstract data types. The order in which the operations are described is not intended to be construed as a limitation, and any number of the described operations can be executed in any order, combined in any order, subdivided into multiple sub-operations, and/or executed in parallel to implement the described processes. The described processes can be performed by resources associated with one or more device(s) 110 or 112, such as one or more internal or external CPUs or GPUs, and/or one or more pieces of hardware logic such as FPGAs, DSPs, or other types of accelerators.

All of the methods and processes described above may be embodied in, and fully automated via, software code modules executed by one or more general purpose computers or processors. The code modules may be stored in any type of computer-readable storage medium or other computer storage device. Some or all of the methods may alternatively be embodied in specialized computer hardware.

Conditional language such as, among others, “can,” “could,” “might” or “may,” unless specifically stated otherwise, are understood within the context to present that certain examples include, while other examples do not include, certain features, elements and/or steps. Thus, such conditional language is not generally intended to imply that certain features, elements and/or steps are in any way required for one or more examples or that one or more examples necessarily include logic for deciding, with or without user input or prompting, whether certain features, elements and/or steps are included or are to be performed in any particular example. Conjunctive language such as the phrase “at least one of X, Y or Z,” unless specifically stated otherwise, is to be understood to present that an item, term, etc. may be either X, Y, or Z, or a combination thereof.

Any routine descriptions, elements or blocks in the flow diagrams described herein and/or depicted in the attached figures should be understood as potentially representing modules, segments, or portions of code that include one or more executable instructions for implementing specific logical functions or elements in the routine. Alternate implementations are included within the scope of the examples described herein in which elements or functions may be deleted, or executed out of order from that shown or discussed, including substantially synchronously or in reverse order, depending on the functionality involved as would be understood by those skilled in the art. It should be emphasized that many variations and modifications may be made to the above-described examples, the elements of which are to be understood as being among other acceptable examples. All such modifications and variations are intended to be included herein within the scope of this disclosure and protected by the following claims.

What is claimed is:

1. A method comprising:
determining an identity of a student user;
presenting a user interface;
obtaining sensor data associated with the student user;
based, at least in part, on the sensor data, determining a state of the student user;
transmitting an indication of the state of the student user to a computer associated with a teacher; and
modifying the user interface based on the state of the student user.

2. A method as recited in claim 1, wherein the state of the student user includes at least one of a cognitive state or an attentive state.

3. A method as recited in claim 1, wherein the sensor data includes biometric sensor data.

4. A method as recited in claim 3, wherein the sensor data further includes one or more of:
keystroke data; or
mouse click data.

5. A method as recited in claim 3, wherein the sensor data further includes facial expression data.

6. A method as recited in claim 1, wherein the state of the student user indicates at least one of:
confusion; or
inattentiveness.

7. A method as recited in claim 1, wherein modifying the user interface based on the state of the student user includes:
when the state of the student user indicates inattentiveness, rendering a prompt to attract attention of the student user.

8. A method as recited in claim 7, wherein the prompt includes at least one of:
a visual prompt; or
an audio prompt.

9. A method as recited in claim 1, wherein modifying the user interface based on the state of the student user includes:
when the state of the student user indicates confusion, rendering a prompt to direct the student user to:
ask a question; or
access a resource.

10. A method comprising:
receiving, from a student computing device, an indication of an identity of a student user of the student computing device;
presenting a user interface that includes a representation of the student computing device;
receiving sensor data associated with the student user of the student computing device;
based, at least in part, on the sensor data, determining a state of the student user of the student computing device; and
modifying the representation of the student computing device in the user interface based on the state of the student user of the student computing device.

11. A method as recited in claim 10, wherein the state of the student user includes at least one of an attentive state or a cognitive state.

12. A method as recited in claim 11, wherein the state of the student user indicates that the student is inattentive.

13. A method as recited in claim 11, wherein the state of the student user indicates that the student is confused.

14. A method as recited in claim 10, wherein the representation of the student computing device in the user interface includes a visual indication of a current state of a student user of the student computing device.

15. A method as recited in claim 10, wherein, the user interface includes representations of multiple student computing devices within an educational environment, the method further comprising:
receiving sensor data associated with each of a plurality of student users of respective student computing devices of the multiple student computing devices;
determining respective states of each of the plurality of student users of respective student computing devices of the multiple student computing devices; and
modifying the user interface such that each respective representation of a student computing device indicates a state of a student user of the respective student computing device.

16. A system comprising:
a teacher computing device associated with a teacher in an educational environment; and
one or more student workstations associated with respective students in the educational environment, wherein a particular student workstation of the one or more student workstations includes:
a student computer;
a student display;
a desk;
a chair; and
one or more sensors,
wherein the teacher computing device is configured to present a user interface that includes respective representations of the one or more student workstations.

17. A system as recited in claim 16, wherein the one or more sensors include any combination of one or more of:
an electroencephalogram sensor;
a functional Near Infrared Spectroscopy (fNIRS) sensor;
a physiological sensor to record one or more of:
a blood pressure reading;
a heart rate;
a galvanic skin response; or
a respiration reading.

18. A system as recited in claim 17, wherein the one or more sensors further include one or more of:
a movement sensor to detect movement of the chair; or
a camera to record student facial expressions.

19. A system as recited in claim 16, wherein the student computer includes:
a student self-monitoring and analysis application configured to:
present a user interface; and
modify the user interface based on a state of the student, wherein the state of the student is determined based, at least in part, on data received from the one or more sensors.

20. A system as recited in claim 16, wherein the teacher computing device is configured to update the user interface to reflect a state of a student associated with each of the respective student workstations.
